(19) United States
(12) Patent Application Publication
(10) Pub. No.: US 2008/0064900 A1
(43) Pub. Date: Mar. 13, 2008

(54) Process for preparing fluorinated acids

(75) Inventors: Nair K. Haridasan, Willamsville, NY (US); Sandra L. Bell, Amherst, NY (US)

Correspondence Address: Honeywell International Inc.;Patent Services Department, 101 Columbia Road, Morristown, NJ, 07962, US

(73) Assignee: Honeywell, Inc.

(21) Appl. No.: 11/900,079

(22) Filed: Sep. 10, 2007

Related U.S. Application Data
(60) Provisional application No. 60/843,965, filed on Sep. 12, 2006.

(51) Int. Cl. C07C 53/21

(52) U.S. Cl. 562/605

(57) ABSTRACT

A process for the preparation a fluorinated acid of the formula R_{f}COOH, wherein the process includes the step of contacting:

(i) a fluorinated alcohol of the formula R_{f}CH₂OH; and (ii) periodic acid;

wherein each R_{f} is independently selected from linear, branched or cyclic hydrocarbyl of 1-12 carbon atoms having 1-25 fluorine atoms and any range there between; and wherein the contacting step is carried out in the presence of a catalyst and optionally in a reaction medium, at a temperature and length of time sufficient to produce the fluorinated acid.

### CROSS-REFERENCE TO A RELATED APPLICATION

The present invention claims priority from U.S. Provisional Application No. 60/843,965, filed Sep. 12, 2006.

### BACKGROUND OF THE INVENTION

1. Field of the Invention

The present invention generally relates to a process for the preparation fluorinated acids from fluorinated alcohols. More particularly, the present invention relates to a process for the preparation fluorinated acids from fluorinated alcohols using periodic acid in the presence of a suitable catalyst.

2. Description of Related Art

Fluorinated acids are important synthetic intermediates in the preparation of industrial and specialty chemicals, such as, insecticides, pharmaceuticals, cosmetics, dyes, and the like. They have also found uses as solvents, catalysts, and lubricants. For example, U.S. Pat. No. 5,736,012 and references cited therein describe preparation and uses of some of these fluorinated acids.

Organic acids are generally prepared by the oxidation of alcohols using oxidizing agents, such as, CrO₃/H₂SO₄, TEMPO/NaOCl or Na₂WO₄/H₂O₂.

Thus, an article in J. Am. Chem. Soc., 1955, 77, 910-914 describes oxidation of CF₃CFHCF₂CH₂OH to CF₃CFHCF₂CO₂H using equimolar amounts of potassium dichromate and concentrated sulfuric acid. U.S. Pat. No. 2,802,028 and an article in J. Am. Chem. Soc., 1958, 80, 6442-6446 describe the preparation of CF₃CFHCF₂CO₂H in low yield (about 23%) by the treatment of a perfluoroolefin, such as, CF₃CF═CF₂, with sodium cyanide and water.

An economically attractive synthesis of pyridiniumfluorochromate and its non-catalytic use in the oxidation of non-fluorinated alcohols to ketone, aldehyde or acids is described in Synthetic Commun., 2004, 22, 4077-4087. Fluorochromate catalyzed periodic acid oxidation of non-fluorinated alcohols, mainly aromatic, is described in J. Fluorine Chem. 2005, 126, 1356-1360.

However, the above procedures and others known in the art have several disadvantages. For example, the sodium cyanide/water method described above involves the use of extremely toxic and poisonous sodium cyanide. Other methods use equally toxic chromium salts. Further, these reagents are used at least in equimolar amounts, but generally in a ratio greater than 1:1 to ensure complete reaction thereby creating problem for scale-up as well as safety and environmental concerns.

The present invention avoids these problems by providing a process for making fluorinated acids from fluorinated alcohols using:

(1) periodic acid in the presence of pyridinium fluorochromate in catalytic amounts; or

(2) periodic acid in the presence of a chromium free catalysts, such as, ruthenium trichloride hydrate (RuCl₃. 3H₂O).

### SUMMARY OF THE INVENTION

The present invention provides a process for the preparation of a fluorinated acid of the formula R_{f}COOH. The process includes the step of contacting:

(i) a fluorinated alcohol of the formula R_{f}CH₂OH; and

(ii) periodic acid;

wherein each R_{f} is independently selected from linear, branched or cyclic hydrocarbyl of 1-12 carbon atoms having 1-25 fluorine atoms and any range there between; and

wherein the contacting step is carried out in the presence of a catalyst and optionally in a reaction medium, at a temperature and length of time sufficient to produce the fluorinated acid.

The present invention provides a process, which is practical and, as such, it is potentially useful commercially.

Unlike the other methods known in the art, the processes described herein are simple one step processes that:

(1) are catalytic;

(2) employ catalysts that contain chromium only in small amounts, i.e., in catalytic amounts; and

(3) employ catalysts that are altogether free of chromium.

These and other benefits of the present invention will become more evident from detailed description of the preferred embodiments that follow.

### DETAILED DESCRIPTION OF THE PREFERRED EMBODIMENTS

Oxidation of alcohols is conveniently carried out by treating them with commercially available periodic acid (H₅IO₆) in the presence of catalytic amount of pyridnium fluorochromate.

For example, CF₃CH₂CF₂CO₂H can be prepared from CF₃CH₂CF₂CH₂OH in 75% yield according to equation (1):

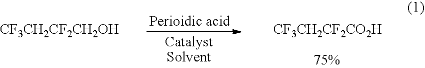

The reaction can be described by the general equation:
R_{f}CH₂OH→R_{f}CO₂H

wherein R_{f} is a fully or partially fluorinated hydrocarbyl group.

Preferably, the R_{f} group is a perfluorohydrocarbyl group of 3-25 fluorine atoms and preferably, 3-13 fluorine atoms.

Preferably, each R_{f} is selected from linear, branched or cyclic hydrocarbyl of 1-6 carbon atoms, wherein each R_{f} contains 1-13 fluorine atoms and any range there between.

More preferably, each R_{f} is selected from linear, branched or cyclic hydrocarbyl of 3-6 carbon atoms.

In a preferred embodiment of the present invention, R_{f} is selected from:

—CF₃CH₂CF₂—, CF₃CH₂CFH—, CF₃CH₂CF₂CH₂—, and CF₃(CF₂)₅CH₂—.

In a preferred embodiment of the present invention, the fluorinated alcohol is selected from:

CF₃CH₂CF₂CH₂OH, CF₃CH₂CFHCH₂OH, CF₃CH₂CF₂CH₂OH, CF₃(CF₂)₅CH₂CH₂OH, and a mixture thereof.

In a preferred embodiment of the present invention, the fluorinated acid is selected from:

CF₃CH₂CF₂COOH, CF₃CH₂CFHCOOH, CF₃CH₂CF₂COOH, CF₃(CF₂)₅CH₂COOH, and a mixture thereof.

Preferably, the fluorinated alcohol is a mixture of at least two alcohols and the fluorinated acid is a mixture of at least two acids.

Preferably, the periodic acid has a concentration of from at least about 95 wt % to about 100 wt %. More preferably, the periodic acid has a concentration of at least about 99 wt %.

Typically, the periodic acid used is from about 1 to about 4 moles and preferably, 1-2 moles per mole of the alcohol, and the catalyst is from about 0.1 to about 5 mole % per mole of the alcohol, preferably from about 0.1 to about 2 mole % of the alcohol.

The requisite alcohols can be made by the procedures described in U.S. Pat. No. 6,673,976 B1 or they can be obtained commercially.

Preferably, the catalyst is selected from pyridinium fluorochromate, ruthenium(III) chloride, and a mixture thereof.

Preferably, the ruthenium(III) chloride is in the form of a trihydrate having the formula RuCl₃.3H₂O and is added at a loading of from about 0.01 mole % to about 10 mole % of the fluorinated alcohol. More preferably, the catalyst is in an amount from about 0.1 mole % to about 2 mole % of the alcohol.

In a typical procedure, preferably the alcohol is added to a mixture of periodic acid and acetonitrile followed by the catalyst and stirred at ambient temperature (25-30° C.) for about 3 hours. The resulting reaction mixture is then filtered, extracted with an organic solvent, such as, ether or ethyl acetate.

The organic extract is then washed with water, treated with a drying agent such as sodium sulfate and filtered. Removal of all solvents from the filtrate at reduced pressure affords the acid, which is typically greater than 95% pure by GC analysis.

Distillation at higher temperature may occasionally cause appreciable decomposition. Therefore, it is generally avoided. However, further purification of the acids can be accomplished by careful distillation at low temperatures under reduced pressure.

The acids are very hygroscopic. In some cases, on storing the acids at room temperature results in slow change in color from colorless to brown. However, despite the color change, a GC analysis shows that there is little or no decomposition or appreciable loss of the acid accompanying the discoloration.

In the practice of the process of the present invention, the step of contacting is preferably carried out at a temperature from about 0° C. to about 50° C. and more preferably from about 5° C. to about 30° C.

In the practice of the process of the present invention, the step of contacting is preferably carried out for a length of time from about 10 minutes to about 48 hours, more preferably about 20 minutes to about 24 hours, still more preferably about 30 minutes to about 12 hours, still yet more preferably from about 3 to about 6 hours, and most preferably about 3.5 hours.

The process can be either a batch process or it can be a continuous process.

The reactor can further include a reaction medium, such as, a solvent or a mixture solvents. Preferably, polar, non-protic solvents, such as, acetonitrile, dimethylformamide (DMF), dimethylsulfoxide (DMSO), ethylene glycol diether, propylene glycol ether acetate, diglyme, triglyme, tetraglyme, and mixtures thereof.

Preferably, the reaction medium is from about 30 wt % to about 90 wt % of the total weight of the reaction mixture.

The process can further include one or more of the following steps:

(1) cooling the reaction mixture to a sub-ambient temperature to precipitate the fluorinated acid;

(2) extracting the fluorinated acid with a solvent; and

(3) distilling the fluorinated acid under reduced pressure to obtain the fluorinated acid in substantially pure form.

In operation, preferably at least 10 wt % of the reactants are converted to the acid product. More preferably, up to at least 80 wt % of the reactants are converted to the acid products, and most preferably, at least 90 wt % of the fluorinated alcohol reactants are converted to fluorinated acid reactants.

The following non-limiting examples are illustrative of the various embodiments of the present invention. It is within the ability of a person of ordinary skill in the art to select other variables from amongst the many known in the art without departing from the scope of the present invention.

Accordingly, these examples shall serve to further illustrate the present invention, not to limit them.

Experimental Details:

Unless otherwise indicated, all parts and percentages are on a weight basis.

### EXAMPLE 1

### Preparation of 2,2,4,4,4-pentafluorobutyric acid (CF₃CH₂CF₂CO₂H):

Into a 2 Liter, 3-necked round bottomed flask equipped with a temperature probe, nitrogen tee, and septum port was added acetonitrile (700 mL) and periodic acid (46 g, 201.8 mmol) and stirred vigorously for 15 minutes. This reaction mixture was then cooled (˜5° C.) and CF₃CH₂CF₂CH₂OH (14.5 g, 88.4 mmol) followed by pyridinium fluorochromate (0.35 g, 1.8 mmol) were added. A slight exotherm was observed. The ice bath was then removed and content in the flask was stirred vigorously for 3 h at 25° C. under nitrogen purge. The resultant solution was filtered. 100 ml ether was added to the filtrate, mixed well and washed successively with 100 ml brine/water (1:1) solution and saturated aqueous NaHSO₃ solution, dried (Na₂SO₄) and filtered. The filtrate was concentrated on Rotary evaporator at 20-30 mm Hg at 25° C. to afford CF₃CH₂CF₂CH₂CO₂H 12.6 g (yield=80 %) as a liquid.

Further purification was accomplished by filtering through a Celite pad and distillation.

NMR and MS spectral data were consistent with the proposed structure:

GC/MS(70 eV): m/e at 179 for (M+1)⁺ (M=C₄H₃F₅O₂), 133 for (M-CO₂H)⁺;

¹⁹F NMR(CDCl₃) δ=−62.2 (tt, overlaps, 3F, J=Hz), −105.5 (m, 2F) ppm;

¹H NMR(CDCl₃) δ=3.1 (qt, overlaps, 2H, J=Hz), —CO₂H proton 3.5 ppm (exchanges with residual water).

¹H NMR indicated that the acid was hydrated with one mole of H₂O.

### EXAMPLE 2

### Preparation of 2,4,4,4-tetrafluorobutyric acid (CF₃CH₂CFHCO₂H):

As described in Example 1, to a stirred mixture of acetonitrile (300 mL) and periodic acid (23 g, 101 mmol) was added CF₃CH₂CFHCH₂OH (7.3 g, 50.0 mmol) followed by pyridinium fluorochromate (0.35 g, 1.8 mmol) at 5° C. The ice bath was removed and content in the flask was stirred vigorously for 3 h at 25° C. under nitrogen purge. The resultant solution was filtered. 50 ml ether was then added to the filtrate, mixed well and washed successively with 100 ml brine/water (1:1) solution and saturated aqueous NaHSO₃ solution, dried (Na₂SO₄) and filtered. The filtrate was concentrated on Rotary evaporator at 20-30 mm Hg at 25° C. to afford CF₃CH₂CFHCO₂H (3.9 g, Yield=51%). GC indicated the material to be >95% pure.

Spectral data were consistent with the proposed structure:

GC/MS (70 eV): m/e at 161 for (M+1)⁺, 160 (M⁺), (M=C₄H₄F₄O₂), 115 for (M-CO₂H)⁺;

¹⁹F NMR(CDCl₃) δ=−64.8 (m, overlaps, 3F), −193.1 (m, 1F) ppm;

¹H NMR(CDCl₃) δ=8.05 (brs, 1H), (5.1 dm, 1H), 2.76 (m, 2H).

### EXAMPLE 3

### Conversion of CF₃CH₂CF₂CH₂OH to CF₃CH₂CF₂CO₂H with RuCl₃.3H₂O

To a stirred mixture of acetonitrile (100 mL) and periodic acid (11.5 g, 50 mmol) was added CF₃CH₂CF₂CH₂OH (4.0 g, 24.4 mmol) followed by RuCl₃.3H₂O (0.30 g, 1.1 mmol) at ˜5° C. The ice bath was removed and content in. the flask was stirred vigorously for 3 hr at 25° C. under nitrogen purge. The resultant solution was filtered and diethyl ether (50 mL) was added to the filtrate, mixed well, washed successively with 100 ml brine/water (1:1) solution and saturated aqueous NaHSO₃ solution, dried (Na₂SO₄) and filtered. The filtrate was concentrated on Rotary evaporator at 20-30 mm Hg at 25° C. to afford 2.0 g CF₃CH₂CF₂CO₂H (Yield=50%).

The identity of this material was confirmed by GC comparison with an authentic sample prepared by the procedure described in Example 1.

### EXAMPLE 4

### Preparation of CF₃CH₂CH₂CO₂H from CF₃CH₂CH₂CH₂OH:

The CF₃CH₂CH₂CH₂OH was oxidized to CF₃CH₂CH₂CO₂H using the procedure described in Example 1; yield 60%.

Spectral data was consistent with the proposed structure.

### EXAMPLE 5

### Preparation of CF₃(CF₂)₅CH₂CO₂H from CF₃(CF₂)₅CH₂CH₂OH:

The CF₃(CF₂)₅CH₂CH₂OH was oxidized to CF₃(CF₂)₅CH₂CO₂H using the procedure described in Example 1; yield 55%. Spectral data were consistent with the proposed structure.

The present invention has been described with particular reference to the preferred embodiments. It should be understood that variations and modifications thereof can be devised by those skilled in the art without departing from the spirit and scope of the present invention. Accordingly, the present invention embraces all such alternatives, modifications and variations that fall within the scope of the appended claims.

What is claimed is:

1. A process for the preparation of a fluorinated acid of the formula R_{f}COOH, comprising:
contacting:
(i) a fluorinated alcohol of the formula R_{f}CH₂OH; and
(ii) periodic acid;
wherein each R_{f} is independently selected from the group consisting of: linear, branched or cyclic hydrocarbyl of 1 to 12 carbon atoms having 1 to 25 fluorine atoms and any range there between; and
wherein said contacting is carried out in the presence of a catalyst and optionally in a reaction medium at a temperature and length of time sufficient to produce said fluorinated acid.

2. The process of claim 1, wherein said R_{f} is a perfluoro-hydrocarbyl group of 3 to 25 fluorine atoms.

3. The process of claim 1, wherein each R_{f} is selected from the group consisting of: linear, branched or cyclic hydrocarbyl of 1-6 carbon atoms having 1-13 fluorine atoms and any range there between.

4. The process of claim 1, wherein each R_{f} is selected from the group consisting of: linear, branched or cyclic hydrocarbyl of 3-6 carbon atoms.

5. The process of claim 1, wherein R_{f} is selected from the group consisting of
CF₃CH₂CF₂—, CF₃CH₂CFH—, CF₃CH₂CF₂—, and CF₃(CF₂)₅CH₂.

6. The process of claim 1, wherein said fluorinated acid is selected from the group consisting of
CF₃CH₂CF₂COOH, CF₃CH₂CFHCOOH, CF₃CH₂CF₂COOH, and CF₃(CF₂)₅CH₂COOH.

7. The process of claim 1, wherein said fluorinated alcohol is selected from the group consisting of
CF₃CH₂CF₂CH₂OH, CF₃CH₂CFHCH₂OH, CF₃CH₂CF₂CH₂OH, and CF₃(CF₂)₅CH₂CH₂OH.

8. The process of claim 1, wherein said fluorinated alcohol is a mixture of at least two alcohols.

9. The process of claim 8, wherein said fluorinated acid is a mixture of at least two acids.

10. The process of claim 1, wherein said periodic acid has a concentration of from at least about 95 wt % to about 100 wt %.

11. The process of claim 1, wherein said periodic acid has a concentration of at least about 99 wt %.

12. The process of claim 1, wherein said catalyst is selected from the group consisting of: pyridinium fluorochromate, ruthenium(III) chloride, and a mixture thereof.

13. The process of claim 12, wherein said ruthenium(III) chloride is in the form of a trihydrate having the formula RuCl₃.3H₂O.

14. The process of claim 1, wherein said catalyst is added at a loading of from about 0.01 mole % to about 10 mole % of said fluorinated alcohol.

15. The process of claim 14, wherein said catalyst is in an amount from about 0.1 mole % to about 2 mol % of the alcohol.

16. The process of claim 1, wherein said periodic acid is in an amount from about 1 mole to about 4 moles per mole of said alcohol.

17. The process of claim 16, wherein said periodic acid is in an amount from about 1 mole to about 2 moles per mole of said alcohol.

18. The process of claim 1, wherein said contacting step is carried out for a length of time from about 30 minutes to about 12 hours.

19. The process of claim 1, wherein said contacting step is carried out for a length of time from about 3 to about 6 hours.

20. The process of claim 1, wherein said step of contacting is carried out in a reaction medium comprising a polar, non-protic solvent.

21. The process of claim 20, wherein said polar, non-protic solvent is selected from the group consisting of:
acetonitrile, dimethylformamide (DMF), dimethylsulfoxide (DMSO), ethylene glycol diether, propylene glycol ether acetate, diglyme, triglyme, tetraglyme, and mixtures thereof.

22. The process of claim 1, further comprising at least one step selected from the group consisting of
cooling said reaction mixture to a sub-ambient temperature to precipitate said fluorinated acid;
extracting said fluorinated acid with a solvent; and
distilling said fluorinated acid under reduced pressure to obtain said fluorinated acid in substantially pure form.
